(19) United States
(12) Patent Application Publication
(10) Pub. No.: US 2001/0001788 A1
(43) Pub. Date: May 24, 2001

(54) Therapeutic composition for allergic dermatitis

(76) Inventors: Toshio Satoh, Tokushima-city (JP); Yutaka Mizushima, Tokyo (JP); Yasuo Kosaka, Matudo-city (JP)

Correspondence Address: PRICE HENEVELD COOPER DEWITT & LITTON, 695 KENMOOR, S.E., P O BOX 2567, GRAND RAPIDS, MI, 49501, US

(21) Appl. No.: 09/747,577

(22) Filed: Dec. 22, 2000

(30) Foreign Application Priority Data
Aug. 18, 1999 (JP) 231048/1999

(51) Int. Cl. A61K 31/70; A01N 43/04

(52) U.S. Cl. 514/25

(57) ABSTRACT

A therapeutic composition for allergic dermatitis or other allergic skin disorders, such as atopic dermatitis. The composition contains an aqueous solution of a naturally occurring macromolecular substance which exhibits both antihistaminic activity and anti-allergic activity. Typically, the aqueous solution of a naturally occurring macromolecular substance is an aqueous solution of chitosan, preferably squid chitosan, having a neutral pH. A method for treating allergic dermatitis including applying the above composition to an affected portion of the subject is also disclosed.

### BACKGROUND OF THE INVENTION

1. Field of the Invention

This invention relates to a therapeutic composition for allergic dermatitis and other allergic skin disorders comprising a naturally occurring macromolecular substance.

2. Related Art

Recently, the incidence of allergic dermatitis, typically atopic dermatitis, has dramatically increased not only in infants but also in adults. Such allergic dermatitis has no single cause, and the causes are increasing in number due to antigen diversity and differences in immune responses of individuals. In particular, the incidence of atopic dermatitis, which is markedly affecting the quality of life (QOL) of children and youths, has been increasing steadily. Medical treatments that have been available for such atopic dermatitis and hay fever are summarized under the three headings below.

Use of steroid ointments

While such ointments have been the first choice of medication used to properly treat these disorders, patients are becoming aware of strong side effects, such as tenderness of the skin and mucous membranes, and increasing numbers of people are abstaining from their use.

Use of histamine-release inhibitors

These drugs attempt to inhibit the release of histamines in the skin, one of the major causes of these disorders. A typical example of such a drug is tranilast (brand name: RIZABEN). However, this group of drugs is not very effective and therefore is not widely used.

Use of antihistamine drugs

These drugs inhibit the actions of histamines, the major cause of atopic dermatitis and hay fever. The most widely used ones combine the actions of an antihistamine and a histamine-release inhibitor. While this is the most commonly used group of drugs for atopic dermatitis and hay fever, serious side effects have been known to occur, although not as serious as those associated with steroid drugs. The most common side effects; i.e., drowsiness, headaches, and dribbling, affect the nervous system, and such effects on the nervous system are peculiar to drugs which are absorbed into the bloodstream.

In view of the foregoing, the present invention aims to provide a therapeutic composition for allergic dermatitis, typically atopic dermatitis, having virtually no side effects.

Japanese Patent Application Laid-Open (kokai) No. 110634/1997, whose inventor is one of the present inventors, and which is incorporated herein by reference, discloses a technique for dissolving chitosan in water in a substantially neutral pH range. This publication discusses how chitosan dissolves in water in an acidic environment, how chitosan is virtually insoluable in a neutral environment, how almost all its physiological activity is exhibited in a neutral environment, and that it is extremely important to dissolve chitosan in water when it is to be used for its physiological activity. The publication also discusses the inventor's discovery that in the presence of an organic acid buffer solution, squid chitosan dissolves in water under certain conditions. The publication discusses skin-protecting ointments that use the moisture-retaining property of chitosan; that is, it discusses the moisture-retaining property of chitosan, and discusses the discovery of a skin protection agent that provides no stimulation when applied to the skin, is pleasant to use, and does not induce an allergic reaction.

The publication provides example methods of manufacturing this skin protection agent, but because the best mode of use is a lotion when the agent is to be applied to allergic dermatitis, some example methods for manufacturing lotions from the above publication are reiterated below.

Lotion 1

Glutamic acid (0.5 g) was dissolved in 100 ml of water, and 1 g of squid chitosan (percent deacylation: 91%) was dissolved in the resultant solution. A lotion was manufactured (viscosity of 250 CP, 20° C.) by adding a 1N sodium hydroxide solution to the solution and agitating until a pH of 5.75 was attained.

Lotion 2

Sodium lactate (0.3 g) was dissolved in 100 ml of water, and 1 g of squid chitosan (percent deacylation: 91%) was dispersed in the resultant solution. Lactic acid was added thereto dropwise for mixing until a pH of 5.75 was attained. The lotion was produced by continuing to agitate the solution until the chitosan was dissolved (viscosity of 280 cp, 20° C.).

Lotion 3

Squid chitosan (1 g, percent deacylation: 91%) was added to 100 ml of a 0.3 M glycolic acid buffer solution (pH 5.60), and the resultant mixture was agitated until dissolution (viscosity 320 CP, 20° C.).

Upon further research, the inventors discovered that this neutral chitosan solution acts as both an antihistaminic and an anti-allergic substance. Until this discovery, no naturally occurring macromolecular substance had been known to be antihistaminic. When an aqueous chitosan solution is applied to the skin or to the mucous membranes, substantially no chitosan is absorbed into the bloodstream, because chitosan is a macromolecular substance having a molecular weight on the order of 10⁴. Accordingly, chitosan accumulates in the skin or mucous membranes in which histamines have been released and acts as a long-lasting antihistaminic agent, but it exhibits almost none of the general side effects exhibited by the commonly used antihistamine drugs. Furthermore, chitosan does not exhibit the side effects of steroids. Therefore, chitosan is an extremely effective medicament for allergic dermatitis such as atopic dermatitis.

The present invention was achieved on the basis of this finding.

### SUMMARY OF THE INVENTION

According to a first aspect of the present invention, there is provided a therapeutic composition for allergic dermatitis comprising an aqueous solution of a naturally occurring macromolecular substance which exhibits antihistaminic activity and anti-allergic activity.

Preferably, the naturally occurring macromolecular substance is chitosan.

Preferably, the naturally occurring macromolecular substance is chitosan, and the aqueous solution is neutral.

Preferably, the naturally occurring macromolecular substance is squid chitosan.

Preferably, the naturally occurring macromolecular substance is squid chitosan which has a percent deacylation of 75% or more.

According to a second aspect of the present invention, there is provided a method for treating allergic skin disorders comprising administering an effective amount of a therapeutic composition comprising an aqueous solution of a naturally occurring macromolecular substance which exhibits anti-histaminic activity and anti-allergic activity.

### BRIEF DESCRIPTION OF THE DRAWINGS

FIG. 1 shows the inhibitory activity on histamine-induced contractions in guinea pig intestine; and

FIG. 2 shows the allergic reaction inhibitory activity in skin from the back of a rat.

### DETAILED DESCRIPTION OF THE PREFERRED EMBODIMENTS

Below is described an experiment conducted on the composition of this invention regarding antihistaminic activity, as well as an experiment testing its anti-allergic activity on a rat.

Experiment regarding Antihistaminic Activity

Method

Preparation of chitosan solution: Powdered chitosan (1.0 g) was added to 1.0 g of sodium L-glutaminate dissolved in 47.0 g of water, to thereby prepare a suspension. A separately prepared solution in which 0.5 g of L-glutaminate had been dissolved in 50.0 g of water was added to the suspension, to thereby prepare a 1% chitosan solution.

Chitosan samples: This experiment employed chitosan sample 1 (percent deacylation: 99%), chitosan sample 2 (percent deacylation: 87%), and diphenhydramine as a positive control.

Action on contraction reaction induced by histamine in an ileum sample extracted from a guinea pig: A male Hartley guinea pig (body weight: between 300 and 400 g) was sacrificed after fasting for 24 hours, and its ileum was extracted immediately thereafter. The extracted ileum was preserved in a vat containing a Tyrode solution. The ileum was cut into pieces having lengths between 1 and 1.5 cm, and the pieces were suspended in a Magnus cup filled with 10 ml of a Tyrode solution. A gas mixture (95% O₂, 5% CO₂) was allowed to flow through the cup continuously, and the temperature was maintained at 37° C. One end of each ileum specimen was fixed to the bottom of the cup, and the other end was connected to an isotonic transducer, with the tension of the specimen being set 1 g. The contraction (or relaxation) value was amplified by an input box and recorded onto a recorder. Histamine training was performed after the ileum was allowed to rest in the Magnus cup for 30 minutes. Briefly, histamine was added to the cup in different concentrations, and percent contraction was measured. This procedure was repeated until reproducibility was obtained; that is, until the same contraction rates were obtained for the same amount of histamine. Next, 0.1 ml of the chitosan solution was added to the cup. After the cup was allowed to stand for five minutes, histamine was added in a cumulative manner. A control experiment was also carried out, in which 0.1 ml of a buffer solution was added before addition of chitosan. The pA2 value was calculated by use of the Takayanagi method generally used in pharmacology. Histamine training and cumulative administration: First, 0.1 ml of a 10⁻⁵ gram/ml histamine solution (final concentration: 10⁻⁷ g/ml) was allowed to act on the ileum specimen. Until contraction became constant, a cycle of washing the specimen and administering the histamine solution was performed repeatedly. Next, in a sequential and cumulative manner, 0.1, 0.2, 0.3, and 0.5 ml doses of a 10^{31 6} g/ml histamine solution and then a 0.1 ml dose of a 10⁻⁵ g/ml solution were administered to the ileum specimen.

Results

The pA₂ values (g/μl) of respective samples are as follows.

Chitosan sample 1 (percent deacylation: 99%): 6.94

Chitosan sample 2 (percent deacylation: 87%): 6.53

Diphenhydramine: 7.43

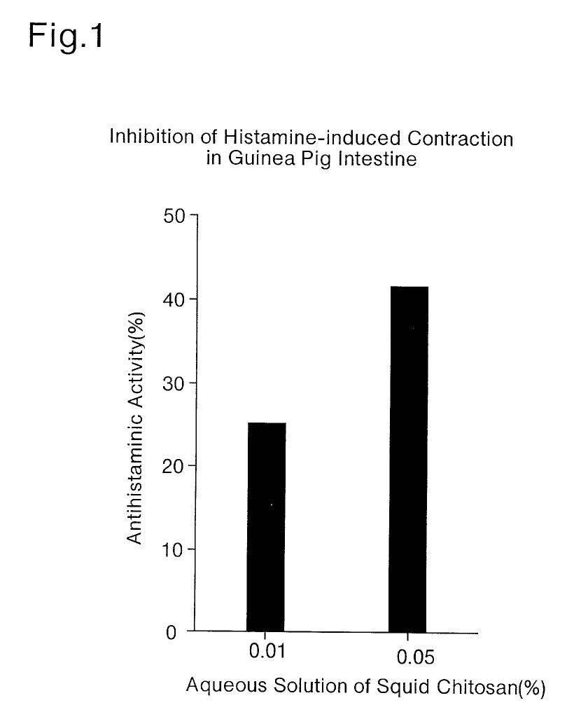

The above results show that the anti-histaminic action of chitosan is one-fifth to one-tenth that of diphenhydramine, and that a higher level of deacylation provides a stronger anti-histaminic action. The inhibitory activity against histamine-elicited contraction in guinea pig intestine is shown in FIG. 1.

Experiment of Anti-allergic Action on Rat

Test Method

Preparation of test solutions: Powdered chitosan (1.0 g) was added to 1.0 g of a sodium L-glutaminate solution dissolved in 47.0 g of water. A solution prepared in advance by dissolving 0.5 g of L-glutaminate in 50.0 g of water was gradually added to the suspension so as to obtain a 1% chitosan solution.

Chitosan sample: Squid chitosan (percent deacylation: 87%) was used as a test substance, and tranilast was used as a positive control.

PCA reaction inhibitory action on skin on the back of rat:

The present experiment was conducted in accordance with the Koda method (Int. Arch. Allergy Appl. Immuno. 87, 251 (1988)). The back of a Wistar male rat (between 200 and 250 g) was clipped, and 0.1 ml of antiserum (DNP-IgE) in dilute physiological saline solution was injected under the skin. The antiserum had already undergone 48 hours of homologous PCA for adjustment of potency. Simultaneously, a physiological saline solution was injected into another part of the rat as a control. After 48 hours, 1 ml of a physiological 5% EvansBlue saline solution containing 1 mg of DNP-BSA protein was injected through the tail vein. After 30 minutes, the rat was decapitated and bled to death. The pigment spots that appeared on the skin of the back were cut out, and the amount of leaked pigment was determined. Briefly, the removed skin was placed in a test tube, and 1 ml of 1 mol/l KOH was added thereto. The test tube was allowed to stand overnight at 37° C. so as to dissolve the skin and to allow elution of the pigment. An acetone-0.6 ml/L phosphate solution (9 ml) was added to the eluted pigment, and, after stirring, the resultant solution was subjected to ten minutes of centrifugal separation at 3,000 rpm. The supernatant was subjected to measurement of absorption at 620 nm, and the amount of pigment was determined by reference to separately prepared calibration curves. The test specimen (0.1 ml) was administered to the sensitized area one hour before administration of the antigen. A site at which no specimen had been administered served as a control and was compared with the drug administration group. Two hours prior to injection of the antigen, tranilast, serving as the positive control, was suspended in 0.2% CMC-Na and orally administered to the rat at a dose of 0.5 ml per 100 g body weight (100 mg/kg).

Results

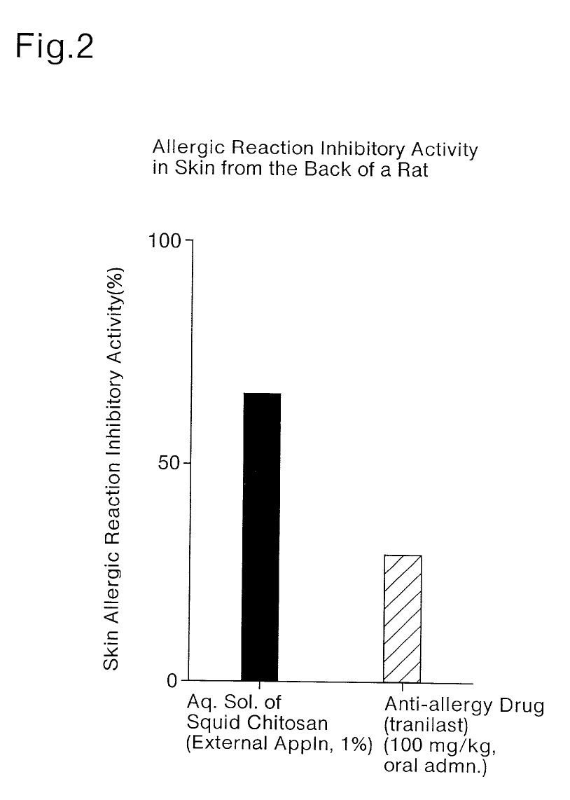

Allergic reaction inhibitory action of a 1% squid chitosan solution applied externally to the skin on the back of the rat is shown in FIG. 2. The solution was roughly two times as effective in inhibiting allergic reactions as compared with the positive control, tranilast.

Some exemplary trials (for amelioration) in which this chitosan lotion was applied on patients are given below.

1. 56-year-old male

Chitosan lotion was applied to a red spot of about 5 cm in diameter on the inside left elbow, where the skin was dry and itchy. Because the itchiness disappeared immediately and the lotion provided a pleasant sensation, application was continued four or five times a day. After three weeks, the dryness and redness had disappeared and the skin had returned to a normal state.

2. 35-year-old male

This subject had applied steroid ointment to atopic dermatitis for several years. However, when the chitosan lotion was applied to good skin at the first signs of dermatitis (when itchiness was initially felt), no smarting occurred and the itchiness stopped. Therefore, application was continued four or five times a day. After three months, the itchiness had completely disappeared and the steroid ointment was no longer necessary.

3. 10-month-old infant

The skin around the crotch of this infant was very red from the wearing of diapers and the infant often cried at night. When the chitosan lotion was applied before the infant went to sleep, the infant slept soundly without crying, presumably because the lotion eased the itching. Application was continued three times a day, and within one month the redness had completely disappeared and the skin had returned to a normal condition.

4. 6-year-old girl

This child suffered from allergic dermatitis which caused a rash of severe itchiness and caused red spots to appear all over her body. Unable to bear the sores caused from scratching and the nighttime itchiness over her body, she would cry at night. When the chitosan lotion was applied all over her body, the itchiness was eased immediately and she slept soundly. Application was continued four or five times a day, and after three months the number of rashes appearing decreased and her skin was almost normal.

5. 75-year-old male

This elderly man had been suffering lack of sleep because of severe itchiness on the back and elsewhere that occurred after bathing and upon going to bed. Therefore, the chitosan lotion was applied on his skin before he went to bed. From the first night, he did not feel itchy and slept soundly.

6. 35-year-old male

This man continually suffered from a runny nose and an itchy nose caused by hay fever. When the chitosan lotion was applied to the inside of his nose by use of a cotton swab, the itchiness was eased and the runniness of the nose was dramatically reduced.

What is claimed is:

1. A therapeutic composition for allergic dermatitis comprising an aqueous solution of a naturally occurring macromolecular substance which exhibits anti-histaminic activity and anti-allergic activity.

2. A therapeutic composition for allergic dermatitis as described in claim 1, wherein the aqueous solution of a naturally occurring macromolecular substance is an aqueous solution of chitosan.

3. A therapeutic composition for allergic dermatitis as described in claim 2, wherein the aqueous solution of chitosan has a pH which is substantially neutral.

4. A therapeutic composition for allergic dermatitis as described in claim 2, wherein the chitosan is squid chitosan.

5. A therapeutic composition for allergic dermatitis as described in claim 3, wherein the chiotosan is squid chitosan.

6. A therapeutic composition for allergic dermatitis as described in claim 4, wherein the squid chitosan has a percent deacylation of 75% or more.

7. A therapeutic composition for allergic dermatitis as described in claim 5, wherein the squid chitosan has a percent deacylation of 75% or more.

8. A meth od for treating allergic dermatitis which comprises applying an effective amount of a composition as recited in claim 1 to an affected part of the skin of a patient in need thereof.

9. A method for treating allergic dermatitis as claimed in claim 8, wherein the allergic dermatitis is atopic dermatitis.

10. A method for treating allergic dermatitis which comprises applying an effective amount of a composition as recited in claim 2 to an affected part of the skin of a patient in need thereof.

11. A method for treating allergic dermatitis which comprises applying an effective amount of a composition as recited in claim 3 to an affected part of the skin of a patient in need thereof.

12. A method for treating allergic dermatitis which comprises applying an effective amount of a composition as recited in claim 4 to an affected part of the skin of a patient in need thereof.
